SEQUENCE LISTING (6 sequences)

SEQ ID NO: 1 - protein, 7 aa, Homo sapiens
SFNRGEC

SEQ ID NO: 2 - protein, 16 aa, Homo sapiens
TVAPTECSSCETPAVT

SEQ ID NO: 3 - protein, 8 aa, Homo sapiens
KSFNRGEC

SEQ ID NO: 4 - protein, 9 aa, Homo sapiens
KTVAPTECS

SEQ ID NO: 5 - protein, 5 aa, Homo sapiens
NRGEC

SEQ ID NO: 6 - protein, 15 aa, Homo sapiens
EGSTVEKTVAPTECS

(19) United States
(12) Patent Application Publication
(10) Pub. No.: US 2024/0418729 A1
(43) Pub. Date: Dec. 19, 2024

(54) METHOD FOR DETECTION AND QUANTIFICATION OF IMMUNOGLOBULIN FREE LIGHT CHAIN DIMERS

(71) Applicant: Rapid Novor, Inc., Kitchener (CA)

(72) Inventors: Mariya LIYASOVA, Kitchener, Ontario (CA); Bin MA, Kitchener, Ontario (CA)

(21) Appl. No.: 18/704,616

(22) PCT Filed: Jun. 16, 2023
(86) PCT No.: PCT/CA2023/050845

§ 371 Date: Apr. 25, 2024

Related U.S. Application Data
(60) Provisional application No. 63/367,941, filed on Jul. 8, 2022.

(51) Int. Cl. G01N 33/68; C12Q 1/37; G01N 1/40; G01N 33/58

(52) U.S. Cl. CPC G01N 33/6848; C12Q 1/37; G01N 1/4044; G01N 33/58; G01N 33/6857; G01N 33/6896; G01N 2333/976

(57) ABSTRACT

Some pathological conditions are accompanied by the increase in dimers of free light chains (FLCs) kappa and/or lambda. For instance, high levels of FLC dimers are found in AL amyloidosis, multiple myeloma, and multiple sclerosis. There is thus a need for sensitive methods that could specifically and quantitatively measure the amount of FLC dimers in a sample. The present application relates to a method for detecting and quantifying immunoglobulin FLC dimers in a sample that comprises subjecting the sample to proteolytic digestion under non-reducing conditions and subjecting the digested sample to mass spectrometry analysis to detect immunoglobulin free light chain dimer peptides. The method may be useful for diagnosis and monitoring of diseases characterized by the aberrant production of FLCs, such as plasma cell discrasias. Light-chain deposition disease (LCDD), autoimmune diseases, chronic kidney disease (CKD), and inflammatory conditions.

### CROSS REFERENCE TO RELATED APPLICATIONS

The present application claims the benefit of U.S. provisional patent application No. 63/367,941 filed on Jul. 8, 2022, which is incorporated herein by reference in its entirety.

### TECHNICAL FIELD

The present invention generally relates to the field of immunoglobulin detection, and more particularly to the detection and quantification of immunoglobulin free light chain dimers in samples.

### BACKGROUND ART

Human immunoglobulins consist of two heavy and two light chains held together by covalent and non-covalent interactions. The light chains are produced by plasma cells in the excess of the heavy chains resulting in the release of the Free Light Chains (FLCs) (1). Based on the amino acid sequence of the constant region, the light chains are divided into two subtypes: κ (kappa) and λ (lambda). The concentration ranges for κ and λ FLCs in normal serum are as follows: κ, 3.3-19.4 mg/L; λ, 5.7-26.3 mg/L; and κ/λ ratio, 0.26-1.65 (2). These concentrations are maintained by the balance between continuous production by the plasma cells and clearance by the kidneys. The half-life of FLCs in serum ranges from 2 to 6 hours (1). FLCs are also present in urine, cerebrospinal fluid (CSF), synovial fluid, tears and saliva.

Some pathological conditions are accompanied by the increase in kappa, lambda or both FLCs. Polyclonal FLC overproduction usually stems from the overall activation of the immune system leading to changes in kappa and/or lambda levels with or without κ/λ ratio changes, while monoclonal FLC overproduction is characterized by the changes in only one type of FLC as well as κ/λ ratio (3). Polyclonal serum FLCs were found to be increased in autoimmune diseases, such as systemic lupus erythematosus (SLE) (4) and inflammatory conditions, such as asthma (5). Infection by some viruses (i.e., HIV) also leads to the increase in the circulating FLCs (6). Overall, the polyclonal FLC overproduction is associated with increased risk of mortality: the hazard ratio for death was found to be 2.07 when the combined FLC concentration was greater than 47.2 mg/L (7).

Of particular interest is the increase in monoclonal FLCs seen in plasma cell dyscrasias (PCDs), such as monoclonal gammopathy of undetermined significance (MGUS), smoldering multiple myeloma (SMM), multiple myeloma (MM), amyloid light-chain (AL) amyloidosis and nonamyloid light chain deposition disease (NALCDD). In MM, high amounts of either kappa or lambda FLCs are produced by malignant plasma cells that proliferate in the bone marrow and cause extensive skeletal pathologies (8). In AL amyloidosis, the specific properties of FLCs make them prone to aggregate and deposit in various tissues leading to organ damage and failure (9). MGUS and SMM are considered benign conditions with the increased risk of developing MM or AL amyloidosis. Serum FLC levels are routinely used for monitoring PCDs (10).

Free light chains exist as monomers, dimers and oligomers. Lambda FLC is particularly prone to form dimers and oligomers. The FLC dimers are stabilized by covalent and non-covalent interactions between the two light chains (11). In kappa LC, the dimer is held together by the disulfide bond between C-terminal cysteines at position 107 (C107), while in lambda LC dimer the disulfide bond is formed between cysteines at position 105 (C105) of constant regions. The conserved residues in the framework region of the variable domain are involved in the noncovalent dimerization of FLCs (12). Under certain pathological conditions, the FLC dimerization changes towards the increased dimer formation. For instance, high levels of FLC dimers were found in AL amyloidosis, MM, and multiple sclerosis (11). The FLC dimerization was not dependent on the total FLC concentration and should thus be considered an independent parameter (13). The pathophysiological role of dimerization is not fully understood, but some studies suggested that the FLC lambda dimers might act as auto-antibodies in severe autoimmune diseases (14).

Free light chain monomer-dimer pattern analysis (FLC-MDPA) found a significant increase in monoclonal FLC dimers in the serum of AL amyloidosis patients. The multivariate analysis showed that the FLC-MDPA could successfully discriminate between AL amyloidosis and benign PCDs, such as MGUS and SMM (15). This finding is of great importance, since currently there is no blood-based test available to diagnose AL amyloidosis. The disease diagnosis relies on painful and laborious biopsy. Moreover, the treatment is initiated only after the occurrence of the organ damage. Thus, the assay that could detect the conversion of benign PCD condition to AL amyloidosis early will prevent the target organ damage. In this respect, FLC dimerization represents a promising biomarker for diagnosis and monitoring of patients with AL amyloidosis.

Similar to the AL amyloidosis patients, the degree of FLC dimerization was significantly higher in MM patients compared to healthy control, MGUS or SMM patients (13). The utility of FLC dimers in diagnosis or monitoring of MM patients is yet to be proven, however the non-invasive nature of such test warrants further investigations.

Multiple sclerosis (MS) is an autoimmune disease characterized by demyelination and neuronal loss. Diagnosis of the disease is challenging since other neurological conditions often present similar clinical manifestations (16). The analysis of CSF for the presence of oligoclonal IgG bands as well as FLCs is now commonly used in the diagnostic work-up of multiple sclerosis (17). FLC dimers were found to be elevated in the CSF of MS patients (18). In 40% of MS patients there was a significant increase in the lambda LC dimers. In other 60% of cases, kappa monomer and dimers were increased. Overall, the analysis of FLC monomer-dimer patterns in CSF could distinguish MS from other neurological conditions with 90% specificity and 96% sensitivity (18). In attempt to develop a non-invasive assay for MS patients, Kaplan et al analyzed the FLC monomer-dimer patterns in saliva of MS patients and healthy individuals. They found that the FLC levels in saliva could distinguish healthy individuals from MS patients (19). Moreover, the salivary FLCs correlated with the relapse/remission status and could thus be used as a tool for monitoring MS disease and response to treatment (20).

FLCs are currently being measured by the nephelometry-based assays Freelite (The Binding Site Group Ltd., Birmingham, UK) and N Latex FLC (Siemens Healthcare Diagnostics Gmbh, Marburg, Germany) (21). The two assays use specific antibodies to detect both monomer and dimer forms of FLCs (22). The assays became a standard practice in MM, AL amyloidosis and increasingly in MS disease screening (23, 24). For instance, the Freelite assay is currently being used to diagnose MM as well as to monitor the response to treatment by differentiating complete response from stringent complete response (21). Despite extensive incorporation into the clinical practice, the nephelometric assays have been criticized for poor accuracy, inability to detect the excess antigen, lot-to-lot variations of antibody reagents, and the lack of consistency between the assays (22, 25).

Considering the broad application of the FLC measurements, mass spectrometry-based techniques were developed (27). The initial report by Bergen et al focused on the characterization of intact FLC monomers and dimers from serum of AL amyloidosis patients (27). Later, the monoclonal immunoglobulin Rapid Accurate Mass Measurement (miRAMM) approach was able to detect and quantify intact monoclonal FLCs in the presence of polyclonal background (28). Moreover, the method allowed the separate analysis of FLC monomers and dimers as well as their glycosylated forms in serum from AL amyloidosis patients (28). Recently, serum FLC immunoenrichment coupled to detection by MALDI-TOF mass spectrometer demonstrated superior sensitivity compared to the Freelite assay (29). However, among the IFE-negative samples with abnormal κ/λ FLC ratios, 24% were not detected by the MALDI-TOF-based approach, indicating that additional pre-analytical optimization is needed. Another limitation is the qualitative nature of the assay (29). An alternative mass spectrometry-based approach measured peptides from the kappa and lambda light chains with selected reaction monitoring (SRM) after the depletion of intact immunoglobulins. This assay allowed the precise quantitation of FLCs down to 3.8 mg/L (kappa) and 2.7 mg/L (lambda) but did not differentiate between monomer and dimer FLC forms (30).

While FLCs are being recognized as biomarkers of plasma cell activity, they lack specificity and might not be adequate for disease monitoring (3). FLC increase due to infection is indistinguishable from that due to the autoimmune disease flare. For example, the FLC levels in subjects with active infection were comparable to those found in SLE patients with a disease flare (31). In the case of AL amyloidosis, FLC measurements by nephelometry might be imprecise due to the aberrant structure of the LCs characteristic to the condition. Moreover, the FLC assay cannot distinguish between malignant conditions such as MM and AL amyloidosis requiring treatment and benign changes, such as MGUS and SMM. On the other hand, measuring FLC dimer in AL amyloidosis patients was found to be specific and sensitive tool in the diagnostic work-up of the disease (15). Importantly, the FLC dimerization was increased even when the total FLC measurements or κ/λ ratio were normal (13). Thus, the sensitive method that could specifically measure the amount of FLC dimers would potentially aid in the diagnosis and monitoring of such conditions as AL amyloidosis, MM and MS. Moreover, quantitative assay is more suitable to monitor the therapeutic efficacy and better at predicting the prognosis.

There is thus a need for sensitive methods that could specifically and quantitatively measure the amount of FLC dimers in a sample, which could be useful for the diagnosis, prognosis, and/or monitoring of conditions associated with FLC overproduction such as AL amyloidosis, MM and MS.

The present description refers to a number of documents, the content of which is herein incorporated by reference in their entirety.

### SUMMARY

In various aspects and embodiments, the present disclosure provides the following items 1 to 27:
1. A method for detection of immunoglobulin free light chain (FLC) dimers in a sample comprising:
   (a) subjecting the sample to proteolytic digestion under non-reducing conditions, thereby obtaining a digested sample; and
   (b) subjecting the digested sample to mass spectrometry analysis to detect immunoglobulin free light chain dimer peptides, wherein the detection of immunoglobulin free light chain dimer peptides is indicative of the presence of immunoglobulin free light chain dimers in the sample.
2. The method of item 1, wherein the sample is a serum sample, cerebrospinal fluid sample or saliva sample.
3. The method of item 1 or 2, wherein the sample is suspected to contain immunoglobulin free light chain dimers.
4. The method of any one of items 1 to 3, wherein the immunoglobulin free light chain dimers comprise dimers of kappa light chains.
5. The method of any one of items 1 to 3, wherein the immunoglobulin free light chain dimers comprise dimers of lambda light chains.
6. The method of any one of items 1 to 5, wherein subjecting the sample to proteolytic digestion comprises contacting the sample with at least one endoprotease.
7. The method of item 6, wherein the at least one endoprotease comprises trypsin, chymotrypsin, LysC, LysargiNase, or any combination thereof.
8. The method of any one of items 1 to 7, wherein the method further comprises subjecting the sample to a denaturating step prior to the proteolytic digestion.
9. The method of item 8, wherein the denaturating step comprises contacting the sample with urea.
10. The method of item 9, wherein the denaturating step comprises contacting the sample with urea at a concentration of at least 4M for at least 30 minutes.
11. The method of any one of items 1 to 10, wherein the method further comprises contacting the sample with a cysteine-modifying agent prior to the proteolytic digestion.
12. The method of item 11, wherein the cysteine-modifying agent comprises N-ethylmaleimide (NEM).
13. The method of any one of items 1 to 12, wherein the mass spectrometry is liquid chromatography coupled to parallel reaction monitoring.
14. The method of item 13, wherein the mass spectrometry analysis is conducted on a quadrupole orbitrap mass spectrometer.
15. The method of any one of items 1 to 14, wherein the immunoglobulin FLC dimers are measured by spiking in a known amount of synthetic peptides containing a specific label.
16. The method of item 15, wherein the specific label comprises a heavy isotope-labeled amino acids.
17. The method of any one of items 1 to 16, further comprising enriching the sample in kappa and/or lambda light chains prior to subjecting the sample to proteolytic digestion.
18. The method of item 17, wherein said enriching comprising contacting the sample with one or more matrices that bind the kappa and/or lambda light chains; and eluting the kappa and/or lambda light chains bound to the one or more matrices. 25
19. The method of any one of items 1 to 18, wherein the immunoglobulin free light chain dimer peptides are

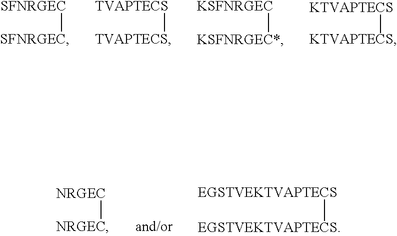

20. The method of any one of items 1 to 19, wherein the sample is a biological sample from a subject suffering from a disease or condition associated with the production of FLCs.
21. The method of item 20, wherein the disease or condition is an autoimmune disease, an inflammatory condition, a viral infection, or a plasma cell dyscrasia (PCD).
22. The method of item 21, wherein the PCD is monoclonal gammopathy of undetermined significance (MGUS), smoldering multiple myeloma (SMM), multiple myeloma (MM), amyloid light-chain (AL) amyloidosis or nonamyloid light chain deposition disease (NALCDD).
23. The method of item 20, wherein the sample is a serum sample from a human subject with multiple myeloma.
24. The method of item 23, wherein the method further comprises quantifying the amount of immunoglobulin FLC dimers in the sample, and wherein the amount of immunoglobulin FLC dimers is correlated to tumor burden.
25. The method of item 20, wherein the sample is from a human subject suffering from or suspected of suffering from multiple sclerosis.
26. The method of item 25, wherein the sample is a cerebrospinal fluid sample
27. The method of item 25, wherein the method comprises comparing the amount of immunoglobulin FLC dimers in the sample to the amount of immunoglobulin FLC dimers in a control sample from a healthy subject.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A and 1B depict MS/MS spectra that confirm the identity of the kappa FLC dimer peptide. FIG. 1A: The fragmentation pattern of m/z 541.22 precursor identifies kappa FLC peptide SFNRGEC (SEQ ID NO:1) linked by the disulfide bond. Recombinant kappa FLC was treated with N-Ethylmaleimide (NEM), digested with LysC and analyzed with LC-MS/MS on Q Exactive mass spectrometer. FIG. 1B: Synthetic dimeric peptide SFNRGEC was analyzed with LC-MS/MS as a positive control. The fragmentation pattern of the m/z 541.22 precursor is identical to that shown in FIG. 1A. The spectra were annotated with pLabel tool of the pLink software (32).

FIGS. 2A and 2B depict MS/MS spectra that confirm the identity of the lambda FLC dimer peptide. FIG. 2A: The fragmentation pattern of m/z 806.35 precursor corresponds to the lambda FLC peptide TVAPTECS (SEQ ID NO:2) linked by the disulfide bond. Recombinant lambda FLC was treated with NEM, digested with LysC and analyzed with LC-MS/MS on Q Exactive mass spectrometer. FIG. 2B: Synthetic dimeric peptide TVAPTECS was analyzed with LC-MS/MS as a positive control. The fragmentation pattern of the m/z 806.35 precursor is identical to that shown in FIG. 2A. The spectra were annotated with pLabel tool of the pLink software (32).

FIGS. 3A-3D depict the extracted ion chromatograms for the FLC dimer peptides. Typical peak shapes of the synthetic kappa FLC dimer peptide SFNRGEC (FIG. 3A), dimer peptide SFNRGEC produced by the LysC digestion of serum spiked with 125 mg/L rFLC kappa (FIG. 3B), synthetic lambda FLC dimer peptide TVAPTECS (FIG. 3C), dimer peptide TVAPTECS produced by the LysC digestion of serum spiked with 125 mg/L rFLC lambda (FIG. 3D). Retention times in minutes and mass accuracy in ppm of the most intense fragment ion are shown above the peak. The images were generated and analyzed with Skyline 20 software (33).

FIGS. 4A-B depict the dilution curves for rFLC kappa (FIG. 4A) and lambda (FIG. 4B) spiked into normal human serum. The data were generated with the PRM assay on the Q Exactive mass spectrometer. The dimer peptides SFNRGEC and TVAPTECS produced by the LysC digestion under non-reducing conditions were measured for kappa and lambda rFLC, respectively. The graphs show the linear dependence of the peptide peak area and the spiked-in rFLC concentration. The goodness of fit was assessed with the R² (shown for each graph).

FIG. 5 depicts the dilution curve for lambda rFLC spiked into normal human serum. The data were generated with the PRM assay on the Q Exactive mass spectrometer. The dimeric peptide KTVAPTECS produced by the LysargiNase digestion under non-reducing conditions was analyzed in the presence of the normal human serum background. The graph shows the linear dependence of the m/z 623.3 peak area and the spiked-in rFLC lambda concentration. The R² shows the goodness of fit to the linear model.

FIG. 6 depicts the peak areas of kappa FLC dimer peptide SFNRGEC (light grey bars, SEQ ID NO: 1) and lambda FLC dimer peptide TVAPTECS (dark grey bars, SEQ ID NO:2) produced by the LysC digestion of control serum in 4 M or 8 M Urea for 1, 4 or 22 hours as indicated on the X-axis. Mean values±SD are shown.

FIG. 7. depicts the normalized peak areas of kappa FLC dimer peptide SFNRGEC (light grey bars) and lambda FLC dimer peptide TVAPTECS (dark grey bars) produced by the LysC digestion of control serum, and serum from MM patients 1916 (IgG Kappa M-protein) and 2666 (IgA Lambda M-protein). Two peptides from human serum albumin were used as internal standards for target peptides signal normalization. Mean values±SD are shown.

FIG. 8. depicts the normalized peak area of kappa FLC dimer peptide SFNRGEC (light grey bars) and lambda FLC dimer peptide TVAPTECS (dark grey bars) produced by the LysC digestion of control serum, control serum enriched for kappa LC with kappa affinity and protein L resin, and control serum enriched for lambda LC with lambda affinity resin. The fold increase of the mean peak area compared to the control value is shown for each enrichment condition.

FIG. 9. depicts the dilution curves for kappa and lambda SIL peptides spiked into the normal human serum. The SIL dimeric peptides SFNRGEC with heavy arginine (circles) and TVAPTECS with heavy valine (squares) were quantified at m/z 547.89 and 812.36, respectively. The graph shows the linear dependence of the SIL peptide peak area and the spiked-in concentration. The goodness of fit was assessed with the R² (shown for each curve).

FIG. 10. depicts lambda FLC dimer monitoring curve for MM patient 2666. The absolute concentrations in mg/L of lambda FLC (Y-axis) were calculated using spiked-in lambda dimeric SIL peptide. Time since the diagnosis in months is shown on the X-axis. The table below the graph lists the lambda FLC dimer concentration, response to treatment, and the M-protein amount measured by serum protein electrophoresis (SPEP). CR denotes complete response to Dexamethasone/Bortezomib/Doxorubicin treatment. PR denotes partial response to treatment, while R denotes the relapse. NQ denotes the non-quantifiable data.

FIG. 11. depicts the amount of kappa FLC dimer (light grey bars) and lambda FLC dimer (dark grey bars) measured in control serum, control saliva, and control CSF by absolute quantification method with spiked-in SIL dimeric peptides. Mean concentration values in mg/L are shown on Log scale on Y-axis.

FIG. 12. depicts the amount of kappa FLC dimer (light grey bars) and lambda FLC dimer (dark grey bars) measured in control CSF, CSF from multiple sclerosis patients, and CSF from patients with other neurological conditions (non-MS, as indicated on the X-axis) by absolute quantification method. The asterisks * indicate the values at least 1.85-fold higher relative to the corresponding values in control #1. Patient IDs shown on X-axis correspond to the patient IDs in the first column of Table 2. Mean concentration values in mg/L are shown on Log scale on Y-axis.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the molecular biology, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The method reported herein relies on the detection and quantification of unique fragments from FLC dimers in samples such as human biological fluids. The assay involves enzymatic digestion of the sample under non-reducing conditions prior to sample analysis. The small FLC dimer fragments monitored in the assay may be detected and quantified with great sensitivity and precision.

The present disclosure provides a method for detection and quantification of immunoglobulin free light chain dimers in a sample comprising: (a) subjecting the sample to proteolytic digestion under non-reducing conditions, thereby obtaining a digested sample; and (b) subjecting the digested sample to mass spectrometry analysis to detect and quantify immunoglobulin free light chain dimer peptides, wherein the detection of immunoglobulin free light chain dimer peptides is indicative of the presence of immunoglobulin free light chain dimers in the sample.

The present disclosure provides a method for identification and quantification of disulfide-bound free light chain dimers in samples such as biological fluids. The method includes 1) collection of the sample (e.g., biological fluid) from the subject, 2) proteolytic digestion of the FLCs with the enzyme under non-reducing conditions to produce the FLC dimer peptides, 3) mass spectral analysis to identify and quantify the FLC dimer peptides.

The term “non-reducing conditions” as used herein means that the digestion is performed under conditions that maintain the disulfide bridges between the light chains, e.g., in the absence of reducing agents such as beta-mercaptoethanol (β-ME), dithiothreitol (DTT) or tris (2-carboxyethyl) phosphine (TCEP).

The method disclosed herein may be used to detect kappa LC dimers and/or lambda LC dimers. In some embodiments, the FLCs may be denatured prior to the proteolytic cleavage. For example, the FLC can be denatured by submitting the sample to heat or by treatment with a denaturating agent such as urea or guanidine hydrochloride. In an embodiment, the sample is treated with urea at a concentration of at least 4M, preferably at least 6M, for example 8M. The treatment may be performed for a time sufficient to denature the sample, for example at least 30 minutes or 1 hour.

In some embodiments, the method comprises the chemical modification of free sulfhydryl groups of cysteines prior to proteolytic cleavage. For example, the free sulfhydryl groups of cysteines may be modified by treatment with agents such as N-Ethylmaleimide (NEM) or iodoacetamide. In some embodiments, the chemical modification is done by NEM to protect the free sulfhydryl groups from random oxidation processes (i.e., disulfide shuffling). In some embodiments the method does not include any chemical modification.

In some embodiments the method comprises isolation or enrichment of FLCs prior to proteolytic cleavage. For example, the isolation of FLCs may be achieved by any suitable protein purification technique such as affinity chromatography, size-exclusion chromatography, ion-exchange chromatography, hydrophobic interaction chromatography (HIC), Melon Gel, and/or gel electrophoresis. In another embodiment, the FLCs may be enriched using a matrix that specifically binds to FLCs. Matrices that can specifically binds kappa and/or lambda light chain are available commercially, e.g., CaptureSelect™ KappaXP Affinity Matrix and CaptureSelect™ LambdaXP Affinity Matrix from ThermoFisher.

The FLCs in the sample may be subjected to proteolytic cleavage with a suitable agent to generate digested peptides. Agents to cleave proteins include chemical agents such cyanogen bromide (CNBr) that cleaves at methionine (Met) residues; BNPS-skatole that cleaves at tryptophan (Trp) residues; formic acid that cleaves at aspartic acid-proline (Asp-Pro) peptide bonds; hydroxylamine that cleaves at asparagine-glycine (Asn-Gly) peptide bonds, and 2-nitro-5-thiocyanobenzoic acid (NTCB) that cleaves at cysteine (Cys) residues, as well as enzymes (e.g., proteases). In an embodiment, the FLCs in the sample is subjected to proteolytic cleavage with any suitable enzyme (e.g., protease) or combinations thereof. Enzymes that may be used to perform the proteolytic cleavage include trypsin, pepsin, chymotrypsin, AspN, LysargiNase, LysC, LysN, GluC, ArgC, Pro/Ala protease, Sap9, KEX2, or any combinations thereof. The digestion may also be performed with a combination of chemical agent(s) and protease(s).

In some embodiments, the FLC dimer peptides are quantified by spiking in the known amount of the labeled synthetic peptides. The synthetic peptides may contain heavy isotope labeled amino acids, substitute amino acids, or chemically modified amino acids in order to create mass difference that distinguishes the labeled peptide from the unlabeled endogenous peptide in the mass spectrum. For example, the arginine, lysine, or valine residues of the synthetic peptides may contain heavy isotopes such as ¹³C, ¹⁵N and/or ²H.

In some embodiments, the method involves determining the ratio of endogenous FLC dimer peptides to serum protein peptides in the sample. Examples of serum proteins are apolipoprotein B, transferrin, and human serum albumin.

In embodiments, the FLC dimer peptide quantification is performed by liquid chromatography-mass spectrometry (LC-MS) coupled to parallel reaction monitoring (PRM). Selected or multiple reaction monitoring (SRM or MRM) can also be used to quantify specific fragment ions from the FLC dimer peptide. The mass spectral analysis may be performed on triple quadrupole mass spectrometer, ion trap mass spectrometer, time of flight, orbitrap or hybrid mass spectrometer.

In an embodiment, the sample is a biological sample such as a biological fluid. In some embodiments, the biological sample is a blood-derived sample (e.g., blood, serum, plasma), urine, saliva, tears, genitourinary secretions, nasal secretions, bronchoalveolar lavage, synovial fluid or cerebrospinal fluid (CSF). The sample may be a biological sample obtained from any animal, including non-human primates or humans. In an embodiment, the sample is a biological sample from a human. In an embodiment, the biological sample is serum. In another embodiment, the biological sample is CSF.

The method disclosed herein may be used to detect and quantify the FLC dimers in samples from subjects suffering from (or suspected of suffering from) any disease characterized by the aberrant production or overexpression of FLCs, such as plasma cell discrasias, autoimmune diseases, chronic kidney disease (CKD), and inflammatory conditions. Examples of plasma cell discrasias characterized by the aberrant production of FLCs include multiple myeloma, plasma cell leukemia (PCL), solitary plasmacytoma (SP), B cell non-Hodgkin's lymphoma (B-NHL), monoclonal gammopathy of undetermined significance (MGUS), smoldering multiple myeloma (SMM), POEMS syndrome, amyloid light chain (AL) amyloidosis, nonamyloid light chain deposition disease (NALCDD) and Waldenstrom's macroglobulinemia. Examples of autoimmune diseases characterized by the aberrant production of FLCs include multiple sclerosis (MS) and systemic lupus erythematosus (SLE). Examples of the inflammatory conditions characterized by the aberrant production of FLCs include asthma.

In some embodiments, the method is used to monitor the patient's response to treatment by analyzing samples from the patient at different time points. The method may include collection of a sample from the patient prior to treatment, collection of one or more samples after the start of the treatment, subjecting the initial and the subsequent sample(s) to the mass spectrometry analysis to quantify the amount of FLC dimers in the samples, and comparing the amounts of the FLC dimers in the samples taken before and after the start of the treatment. The method may be especially useful for monitoring light chain-only multiple myeloma, a condition in which only LC without the heavy chain is produced by the malignant plasma cells.

The method may also be used to predict the relapse (e.g., in multiple myeloma patients) or the disease flare (e.g., in multiple sclerosis patients). By measuring the quantity of FLC dimers in samples from the patient over time, it is possible to predict the relapse if the amount of the FLC dimers starts to increase in samples from the patient. The early detection of relapse can help guide the decision to change the treatment and thus result in a better patient outcome.

The method may be used in combination with other techniques to diagnose some conditions, such as AL amyloidosis and multiple sclerosis, for example by comparing the amount of FLC dimers in the biological fluids of the patient to the amount of FLC dimers in the biological fluids taken from control healthy subjects.

### EXAMPLES

The present disclosure is illustrated in further details by the following non-limiting examples.

### Example 1: Enzymatic Digestion of Recombinant FLCs Produces Unique Dimer Peptides

Kappa FLCs form dimers through the disulfide bonding between the constant region cysteines 107 of each monomer chain. Lambda FLC dimers are formed through the disulfide bonding between the constant region cysteines 105. Thus, the enzymatic digestion of dimerized FLC under the non-reducing conditions can release the unique peptide characteristic of either kappa or lambda FLC dimer.

To find and characterize the FLC dimer peptides, the human recombinant FLC (rFLC) kappa (Pr00115) or rFLC lambda (Pr00116) from Absolute Antibodies were used. The PAGE under non-reducing condition demonstrated the presence of both monomer (25 kDa) and dimer (50 kDa) species in both reagents. In addition, de novo protein sequencing confirmed the identities of the commercial proteins and decoded their full amino acid sequences.

Recombinant FLC kappa or rFLC lambda was first reacted with N-ethylmaleimide (NEM) to block the free sulfide groups and thus prevent the random disulfide bond shuffling. 100 μg of recombinant FLC was reacted with 100 μg of NEM in 0.1 M PBS, pH 7.2 in the presence of 4M Urea for 2 hours at room temperature. The excess of NEM was then removed by Zeba™ Spin Desalting Columns 7 kDa MWCO according to the manufacturer's instructions. 10 μg of NEM-treated FLC was taken for the enzymatic digestion with either LysC, LysargiNase, Trypsin or Chymotrypsin protease (MS grade). The enzyme to protein ratio of 1:50 was used for all proteases. The digestion was performed in 0.1 M Tris Buffer, pH 8.0 for 18 hours at 37° C. The equivalent of 0.5 μg of the digested protein was then loaded on the EV-2001 C18 Evotips (Evosep, Odense C, Denmark) per manufacturer's instructions. The peptides were separated on 15 cm C18 column (PepSep, ReproSil 3 μm C18 beads, 100 μm ID) with the proprietary Evosep gradient of 0.1% FA/ACN for 44 min. The eluted peptides were injected in-line to the Q Exactive™ Hybrid Quadrupole-Orbitrap™ mass spectrometer (ThermoFisher Scientific). For the ionization, stainless steel emitters (ID 30 μm, OD 150 μm, PepSep, Marslev, Denmark) were maintained at 2 kV. DDA mode with the following parameters was used for the peptide selection: the MS spectra were collected with orbitrap resolution of 70000, scan range of 400-2000 m/z, AGC target of 3e6, and max IT of 100 ms. MS/MS scans were performed in centroid mode with orbitrap resolution of 17500, quadrupole isolation window of 2.4 m/z, AGC target of 3e6, max IT of 100 ms and 27% collision energy for HCD fragmentation. The dynamic exclusion was set to 7 s.

The DDA data was submitted to the pLink software for the identification of the disulfide bond linked peptides (32). The pLink analysis was performed with the following parameters: SS linker, 4 missed cleavages, peptide mass range 400-6000, peptide length 3-60, precursor and fragment tolerance of 20 ppm and N-Ethylmaleimide as a variable modification.

Each digestion protease produced the unique kappa and lambda dimer peptide, listed in Table 1. Only symmetrical, specifically cleaved peptides are shown. The identity of each peptide was confirmed by the MS/MS spectrum. Dimeric peptides SFNRGEC (SEQ ID NO:1) and TVAPTECS (SEQ ID NO:2) produced respectively from kappa and lambda FLC by LysC, as well as the KTVAPTECS (SEQ ID NO:4) dimer produced from lambda FLC by LysargiNase digestion were selected for further investigation.

TABLE 1
FLC dimer peptides produced by various digestion enzymes
under non-reducing conditions

| | Kappa FLC | | Lambda | |
|---|---|---|---|---|
| | dimer | Kappa | FLC | Lambda |
| | peptide | Pre- | dimer | Pre- |
| | (SEQ | cursor | peptide | cursor |
| Enzyme | ID NO:) | mass | (SEQ ID NO:) | mass |
| LysC | 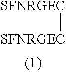 | 1621.65 | 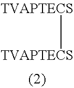 | 1611.69 |
| Lysargi- Nase | 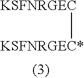 | 1877.84 | 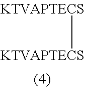 | 1867.88 |
| Trypsin | 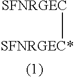 | 1621.65 | 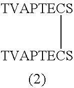 | 1611.69 |
| Chymo- trypsin | 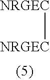 | 1153.45 | 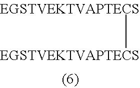 | 3072.39 |

*denotes miscleaved peptide

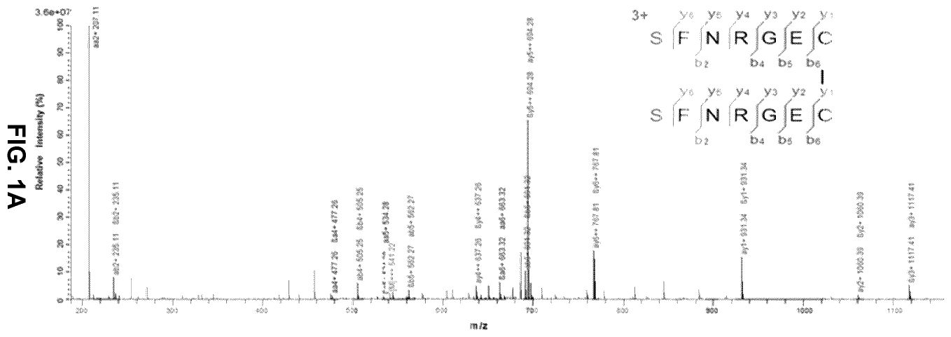

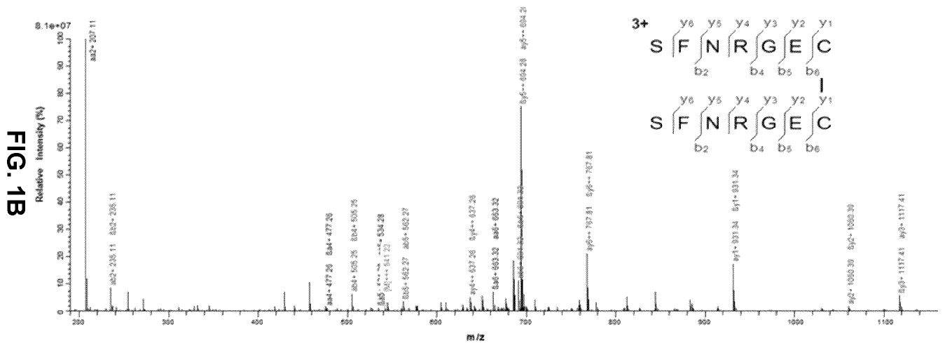

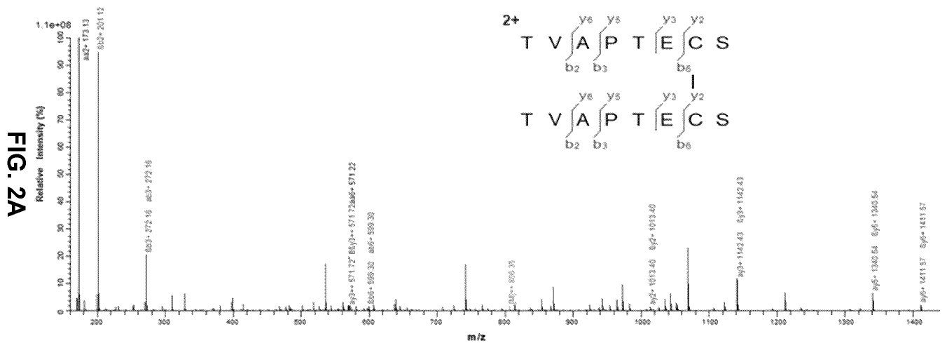

The MS/MS spectrum of precursor at m/z 541.22 confirms the identity of the disulfide-linked peptide SFNRGEC at the C-terminus of kappa FLC (FIG. 1A) produced by the cleavage by the LysC protease. The fragmentation of precursor at m/z 806.35 confirms the identity of the disulfide-linked peptide TVAPTECS (SEQ ID NO:2) produced by the LysC digestion of lambda FLC (FIG. 2A). The spectra of cross-linked peptides were annotated using the pLabel tool of the pLink software. All the major peaks are assigned to the specific a-, b- or y-fragment ions. Note that the y-ions contain the disulfide bond. Other observed peaks represent the internal fragment ions. The identical spectra were obtained when the pure synthetic disulfide-linked peptides SFNRGEC (SEQ ID NO:1) or TVAPTECS (SEQ ID NO:2) were analyzed with mass spectrometry (FIGS. 1B and 2B, respectively).

### Example 2: Quantification of Kappa and Lambda LC Dimers in Spiked-In Control Serum

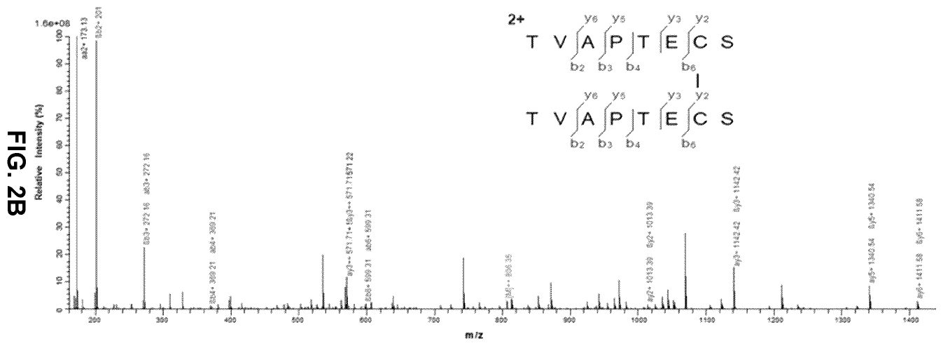

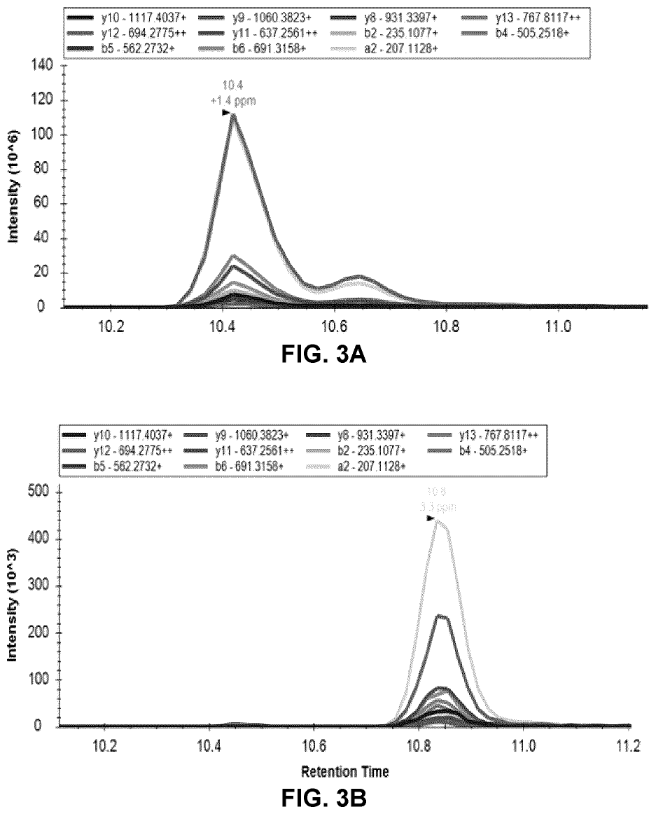

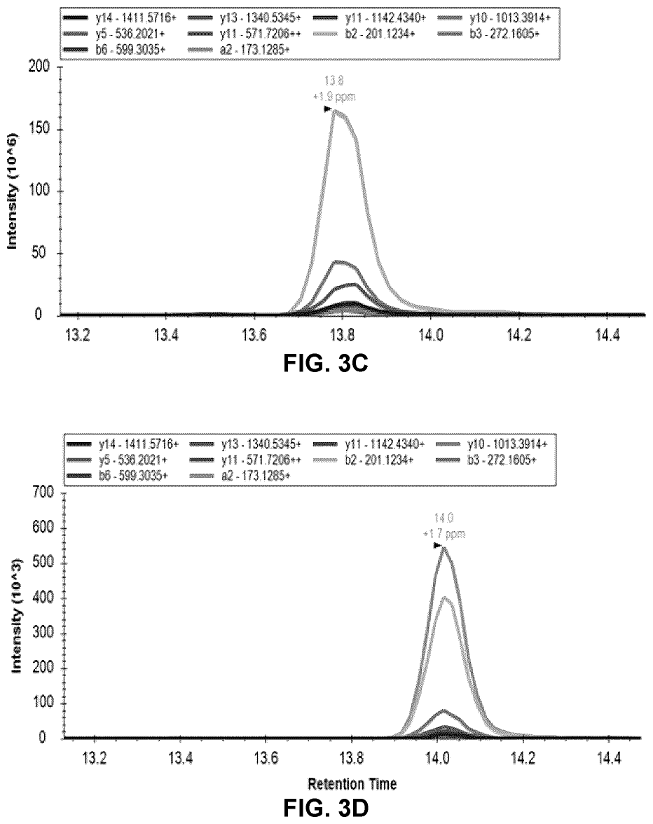

To quantify the dimeric FLC peptides in human serum, the recombinant FLC was dried in the CentriVap and reconstituted in the control human serum (H4522, Sigma-Aldrich) at the final concentration of 2 g/L. The spiked-in serum was then serially diluted into the control serum in 2-fold increments up to the final dilution of 64-fold. Control serum without recombinant FLC served as a negative control (0 g/L). The spiked-in serum was then treated with the equivalent amount of NEM and digested with either LysC or LysargiNase as described in Example 1. The equivalent of 1 μg of the digest was loaded on the Evotips, separated on 15 cm C18 column for 44 min as described above. For dimer peptide quantification targeted MS/MS spectra (PRM) were collected on Q Exactive Orbitrap in centroid mode with the following parameters: orbitrap resolution of 17500, quadrupole isolation window of 2 m/z, AGC target of 3e6, max IT of 100 ms, 27% collision energy for HCD fragmentation. The PRM data were analyzed using Skyline 20 software (33). After LysC digestion, the triply charged parent ion at m/z 541.22 was monitored to quantify the kappa FLC dimer, while the doubly charged parent ion at m/z 806.35 was monitored for the lambda FLC. FIG. 3 shows the typical peak shapes of the pure synthetic dimer peptides SFNRGEC (approximated as SFNRGEC(Oxi)SFNRGEC in Skyline) (FIG. 3A) and TVAPTECS (approximated as TVAPTEC(Oxi)STVAPTECS in Skyline) (FIG. 3C) and of the same peptides produced by the LysC digestion of serum spiked with 125 mg/L rFLC kappa (FIG. 3B) and lambda (FIG. 3D). The ion distribution pattern and retention times were similar for pure dimeric peptide and the dimeric peptide produced by the LysC digestion of the rFLC.

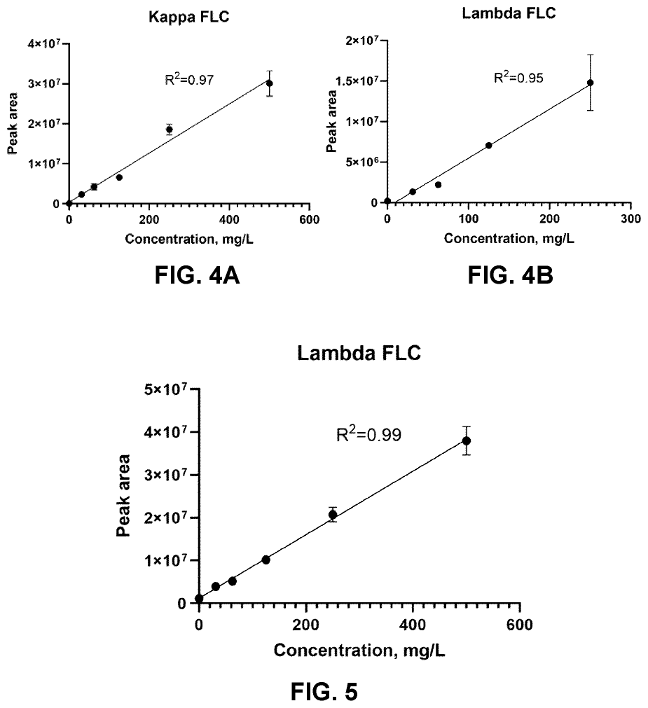

To establish the limit of detection (LOD) and lower limit of quantification (LLoQ), the dilution curves for kappa and lambda dimer peptides were generated. The linear relationship between the peak area and the rFLC concentration was observed for dimeric peptides SFNRGEC (kappa FLC), TVAPTECS (lambda FLC) (FIG. 4) produced by the LysC digestion, as well as for the dimeric peptide KTVAPTECS produced from lambda FLC by the LysargiNase digestion (FIG. 5).

The LOD was defined as the lowest rFLC concentration where the ion distribution pattern was similar to that of the synthetic peptide and the mass error for individual transitions was less than 10 ppm. The LLoQ was defined as the lowest rFLC concentration where calculated values were within 80-120% of the expected values and the coefficient of variation (CV) of duplicate injections was less than 20%.

All the peptides' peak areas showed good linearity R²>0.95 (FIGS. 4 and 5). The LLoQ for kappa dimer was as low as 31.25 mg/L. The lambda dimer could be accurately quantified by monitoring TVAPTECS-TVAPTECS peptide down to 125 mg/L or by monitoring KTVAPTECS-KTVAPTECS peptide down to 62.5 mg/L. The LOD could not be established for kappa or lambda dimer peptides since they were detected in the normal serum without the rFLC. To put these numbers into prospective, it must be considered that the reference ranges for kappa FLC and lambda FLC in healthy individuals are 3.3-19.4 mg/L and 5.7-26.3 mg/L, respectively, and that the median difference between involved and uninvolved FLC concentration (dFLC) in AL amyloidosis is 180 mg/L (2,34). Thus, it may be concluded that the FLC dimer can be quantified with mass spectrometry method with a sensitivity sufficient to detect the differences between healthy and diseased subjects.

### Example 3: FLC Dimers can be Measured in Control Serum and Serum from Patients with Multiple Myeloma

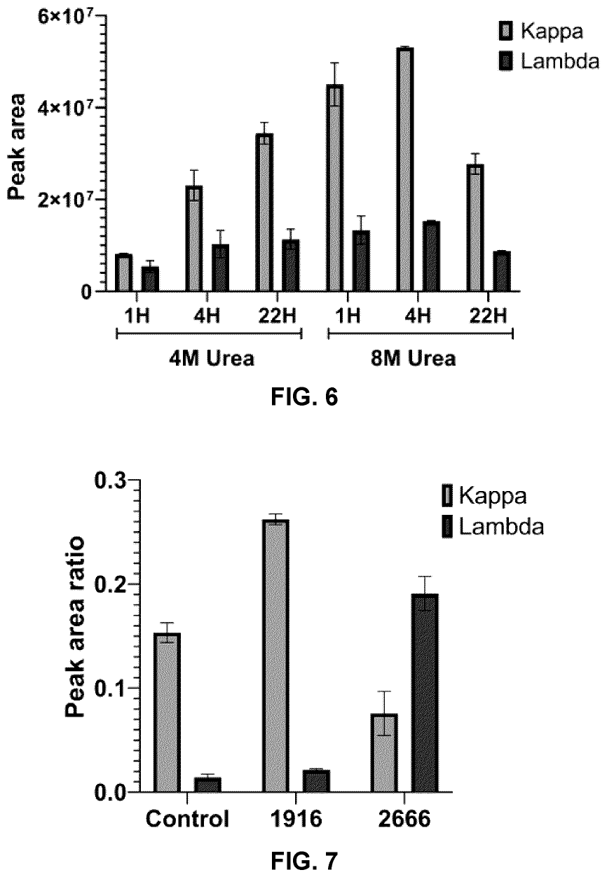

As mentioned in Example 2, free light chain dimers can be detected in control pooled serum (H4522, Sigma). In order to maximize the release of the dimeric peptide by LysC under non-reducing conditions, several digestion parameters were iterated. Namely, the digestion was performed in 4 M and 8 M Urea for either 1, 4 or 22 hours. N-ethylmaleimide (NEM) was not used since the formation of the FLC dimers due to the random disulfide bond shuffling in serum is unlikely. Briefly, control serum H4522 was diluted 12-fold with water to a total protein concentration of 5 μg/μl. 2 μl of diluted serum (10 μg) was then mixed with 32 μl of 0.1 M Tris buffer, pH 8.0 containing 4 or 8 M Urea and 0.2 μg LysC (enzyme to protein ratio of 1:50). The digestion reactions were incubated at 35° C. for 1, 4 or 22 hours, then stopped by acidification with TFA. The equivalent of 0.8 μg of the digested protein was then loaded on the EV2011 Evotip Pure C18 desalting trap columns (Evosep, Odense C, Denmark) per manufacturer's instructions, and separated on 15 cm C18 column with the proprietary Evosep gradient of 0.1% FA/ACN for 44 min. The targeted dimer peptide quantification (PRM) was performed on Orbitrap Exploris 240 Mass Spectrometer in profile mode with the following parameters: orbitrap resolution of 30,000, quadrupole isolation window of 0.6 m/z with the m/z offset of 0.25, standard AGC target, dynamic max IT mode, 27% collision energy for HCD fragmentation. Ions at m/z 541.22 and 806.35 were monitored to quantify kappa and lambda FLC dimers, respectively. The results shown in FIG. 6 indicate that the optimal release of kappa and lambda dimeric peptides was achieved in 8 M Urea in 1 or 4 hours. Shorter reaction time of 1 hour was selected for all future experiments to streamline the procedure.

The optimized digestion conditions were then applied to measure the FLC dimers in serum samples from patients with multiple myeloma (MM), a disease often associated with overproduction the monoclonal immunoglobulin (M-protein) and FLCs. Serum samples from patients with IgG kappa (1916) and IgA lambda (2666) MM were purchased from the Institute for Myeloma and Bone Cancer Research, California, USA. Sample collection was approved by Western Institutional Review Board (WIRB), and each patient was individually consented. The diagnostic samples contained M-protein at 11.7 g/L (1916) and 44.7 g/L (2666) measured by serum protein electrophoresis. FLC measurements were not available. The diluted serum samples were digested with LysC in the presence of 8M urea for 1 hour at 35° C., the digests were then loaded on Evotips and analyzed with Exploris-240 as described above. To reduce run-to run variations, the signal from kappa and lambda FLC dimer was normalized on the signal from 2 LysC peptides from human serum albumin (HSA). The internal standard peptides SLHTLFGDK and TPVSDRVTK were chosen to match the target peptides signal intensities as well as elution profiles. Other serum proteins, such as transferrin, can also be used as endogenous internal standards for FLC dimer signal normalization. The normalized peak areas for kappa and lambda FLC dimers in control and MM serum are shown in FIG. 7. The amount of kappa LC dimers in serum from patient 1916 with IgG kappa M-protein was 1.7-fold higher than the amount measured in control pooled serum (compare light grey bars in FIG. 7). The amount of lambda LC dimers in serum from patient 2666 with IgA lambda M-protein was 13.5-fold higher than the amount measured in control pooled serum (compare dark grey bars in FIG. 7). Interestingly, the amount of kappa FLC dimers in serum of patient 2666 was 2-fold lower than in control serum. The phenomenon of suppression of uninvolved LC, called immunoparesis, is well described in the literature (35).

The data presented in this example clearly demonstrate that the levels of kappa and lambda FLC dimers in serum of patients with MM are different from those in control serum, providing evidence that the assay described herein may be useful for the diagnosis and monitoring of MM.

### Example 4: Enrichment of Kappa and Lambda LC Improves the Detection of Kappa and Lambda FLC Dimers in Control Serum

To improve the signal from FLC dimers and thus increase the sensitivity of detection, kappa and lambda FLCs were separately enriched from the control serum. Briefly, 50 μl to 100 μl of control pooled serum was first depleted from IgG fraction by G-beads according to the manufacturer's instruction (Magne™ Protein G Beads, Promega). The IgG-depleted serum was then incubated with the Capture Select affinity beads specific for kappa (KappaXP) or lambda (LambdaXP) LCs. Separate aliquot of IgG-depleted serum was incubated with protein L agarose, which specifically binds to human kappa LCs. The bound kappa or lambda LCs were eluted in 0.1 M glycine buffer, pH 2.5. The eluates were neutralized by adding 1M Tris buffer, pH 8.0, dried in the CentriVap, reconstituted in 0.1 M Tris buffer, pH 8.0 containing 8 M Urea and digested with LysC (0.2 μg/reaction). The digestion of diluted serum without the enrichment was done in parallel for comparison. The digests were loaded on Evotips and analyzed with Exploris-240 as described in Example 3.

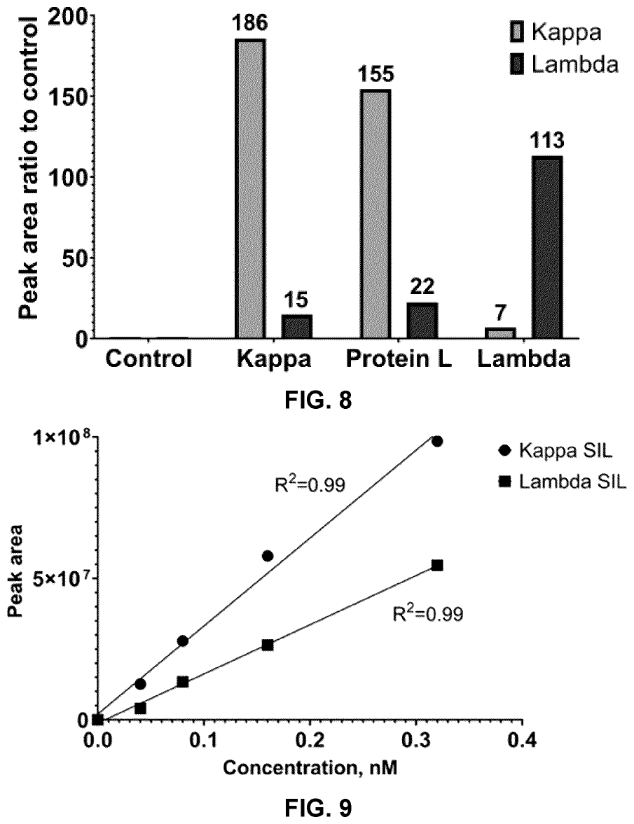

The enrichment of kappa LC from 50 μl of serum with kappa affinity resin resulted in 186-fold increase in kappa FLC dimer signal compared to un-enriched serum (FIG. 8). Comparable enrichment of kappa LC and 155-fold increase in kappa FLC dimer signal was achieved with protein L resin. The enrichment of lambda LC from 100 μl of serum with lambda affinity resin resulted in 113-fold increase in lambda FLC dimer signal compared to un-enriched serum. Surprisingly, lambda affinity resin also non-specifically bound kappa LCs and vice versa. Binding, washing, and elution conditions can be optimized to minimize non-specific binding, as well as to improve targeted enrichment.

The enrichment strategy described in this example can be used to increase the sensitivity of the detection of FLC dimers in serum. In addition, enrichment from other biological fluids such as saliva, tears, CSF and urine can be performed.

### Example 5: Absolute Quantification of Lambda LC Dimers in Serial Samples from Patient with MM

Absolute quantification of FLC dimers can be achieved by spiking the known quantity of stable isotope labeled (SIL) peptides into the serum digest to use as a single point internal calibrant. The SIL dimeric peptide SNFRGEC was synthesized with heavy-isotope labeled Arginine (13C6, 15N4), while the SIL dimeric peptide TVAPTESC was synthesized with heavy Valine (13C5,15N). The synthesis was performed by GenScript, Piscataway, NJ, USA. The peptide identity was confirmed by the mass spectrometry analysis. The peptide net amount was derived from the amino acid (AA) analysis.

The SIL peptides were spiked into control serum digest in increasing quantities. The ions with m/z 547.89 and 812.36 were monitored for kappa and lambda SIL dimeric peptides, respectively. The RT and ion distributions were identical for unlabeled and SIL peptides. FIG. 9 demonstrates the linear relationship between the SIL peptide concentration and the MS peak area (R²=0.99). Importantly, the amount of unlabeled FLC dimer peptides in serum was not affected by the addition of the SIL peptides. Based on the expected peak area of the FLC dimer peptides in control serum samples, the optimal target concentration of SIL peptide was 0.16-0.32 nM.

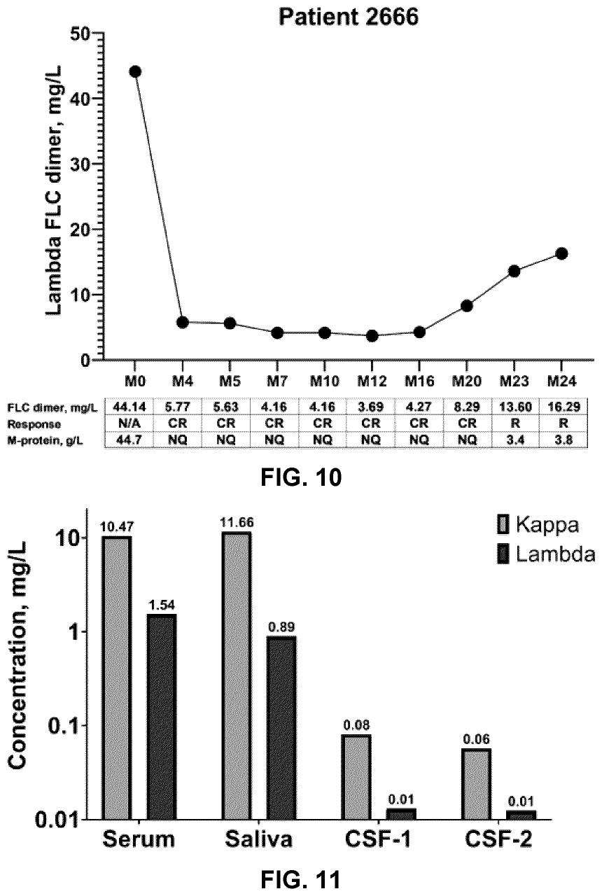

To demonstrate that the SIL-peptides can be used for absolute quantification of LC dimers in serum, MM patient 2666 from Example 3 was selected. Serial serum samples obtained from this patient spanned a period of 2 years during which the patient was treated, reached the complete response (CR) and relapsed (R). The serum samples were digested with LysC in the presence of 8 M Urea as described in Example 3. The digests were then spiked with lambda SIL (0.286 nM final concentration), loaded on Evotips and analyzed with Exloris-240. The acquisition method was modified to include the SIL peptide mass. The peak area of the unlabeled dimeric peptide TVAPTESC was normalized on the peak area of the spiked SIL peptide. Based on the spiked amount of SIL peptide the concentration of lambda dimer was calculated for each time point and plotted vs. time since MM diagnosis. The lambda FLC dimer monitoring curve depicted in FIG. 10 correlates well with the M-protein amount as well as overall disease status at each time point. The initial decrease in lambda dimer amount reflects the response to treatment, while the later increase is consistent with relapse. Moreover, the increase in the FLC dimer amount was observed from M16 to M20, 3 months earlier than the relapse was confirmed by the conventional assays, such as SPEP and IFE. Thus, this example demonstrates the utility of FLC dimer measurement with mass spectrometry in the monitoring of MM disease status and detection of relapse. The assay is even more important for the monitoring of LC-only MM for which very limited options exist in the clinic.

### Example 6: Absolute Quantification of FLC Dimers in Serum, Saliva and CSF

To demonstrate the broad applicability of FLC dimer quantification with MS, the amount of FLC dimers was quantified and compared in various biological fluids, such as serum, saliva, and CSF. Pooled serum (H4522, Sigma) was diluted and digested with LysC as described in Example 3. Saliva was collected from healthy individual by the passive drool method in the morning hours before eating. Freshly collected saliva was centrifuged at 16,000 g prior to analysis. CSF from 2 healthy individuals was purchased form BiolVT, New York, NY, USA. 10 μl of saliva or 20 μl of CSF was dried in the CentriVap, reconstituted in 0.1 M Tris buffer, pH 8.0 containing 8 M Urea and digested with LysC (0.2 μg/reaction) for 1H at 35° C. The digests were diluted and spiked with kappa dimeric SIL peptide SFNRGEC and lambda dimeric SIL peptide TVAPTECS at 0.286 nM and 0.107 nM, respectively. The SIL-spiked digests were loaded on Evotips and analyzed with Exploris-240 as descried in Example 3. The concentrations of kappa and lambda FLC dimers were compared among the three fluids (FIG. 11). Comparable amounts of FLC dimers were present in serum and saliva, while significantly lower amounts were detected in CSF. The relative abundance of kappa dimers to lambda dimers was conserved among the analyzed fluids and ranged from 5:1 to 13:1.

### Example 7: Absolute Quantification of FLC Dimers in Control CSF and CSF from Patients with Multiple Sclerosis

Multiple sclerosis (MS) is often associated with the increased amounts of oligoclonal Igs and FLCs in the CSF (17). Thus, the amounts of FLC dimers in the CSF of control subjects and the CSF of multiple sclerosis patients was quantified and compared. CSF from healthy individuals as well as from patients with multiple sclerosis and other non-MS neurological conditions (stroke, migraine, clinically isolated syndrome and demyelinating disease) were purchased form BioIVT. Table 2 shows patient demographics and clinical data.

TABLE 2
Patient demographics and clinical data

| | | | | Time | |
|---|---|---|---|---|---|
| Patient | | | | with MS | |
| ID | Age | Gender | Diagnosis | (months) | Treatment |
| 1 | 56 | M | None | N/A | None |
| 2 | 36 | F | None | N/A | None |
| 3 | 56 | M | Secondary | 330 | Ocrevus 300 mg |
| | | | progressive MS | | |
| 4 | 56 | M | RRMS, | 75 | Aubagio 14 mg, Gabapentin |
| | | | Hypertension, | | 600 mg, Ibuprofen 600 mg, |
| | | | Hyper- | | Lisinopril 20 mg, Oxycodone |
| | | | cholesterolemia, | | 10 mg, Amlodipine 5 mg |
| | | | Chronic lower | | |
| | | | back pain | | |
| 5 | 79 | F | Moderate Multiple | 413 | Betaseron 0.3 mg, Lantus |
| | | | Sclerosis, Type 2 | | 30U, Escitalopram 10 mg, |
| | | | Diabetes, | | Metoprolol 50 mg, |
| | | | Hypertension, | | Pramipexole 1.5 mg, |
| | | | Osteoarthritis, | | Hydralazine 25 mg, |
| | | | Depression | | Glimepiride 2 mg, Losartan |
| | | | | | 100 mg |
| 6 | 54 | F | Moderate RRMS, | 246 | Aubagio 14 mg, Levetiracetam |
| | | | Depression, | | 750 mg, Citalopram 40 mg, |
| | | | Anxiety, | | Baclofen 10 mg, Amantadine |
| | | | Insomnia | | 100 mg, Zolpidem 10 mg, |
| | | | | | Quetiapine 200 mg |
| 7 | 32 | M | RRMS, Seizures | 0* | Gabapentin 800 mg, Keppra |
| | | | | | 500mg, |
| 8 | 24 | F | RRMS, Asthma, | 0* | None |
| | | | Eczema | | |
| 9 | 36 | F | Multiple Sclerosis, | NR | Breo, Lessina Birth Control, |
| | | | Asthma | | Albuterol, Topiramate |
| 10 | 68 | F | Migraine | N/A | Levoxyl 100 mg, Zomig 5 mg |
| 11 | 26 | F | Clinically Isolated | N/A | None |
| | | | Syndrome (CIS) | | |
| 12 | 55 | F | Demyelinating | Confirmed | None |
| | | | Disease | Negative | |
| | | | | for MS by | |
| | | | | Report | |
| 13 | 66 | F | Stroke, Chronic | N/A | Aspirin 81 mg, Levothyroxine |
| | | | obstructive | | 50 mcg, Omeprazole 40 mg, |
| | | | pulmonary disease, | | Ipratropium 0.5/2.5 mg, |
| | | | Hypertension, High | | Isosorbide 20 mg, Gabapentin |
| | | | cholesterol, | | 300 mg, Atenolol 25 mg, |
| | | | Chronic pain, | | Memantine 10 mg |
| | | | Hypothyroidism | | |

*newly diagnosed;
N/A, not applicable;
NR, not reported;
RRMS, relapsing-remitting multiple sclerosis

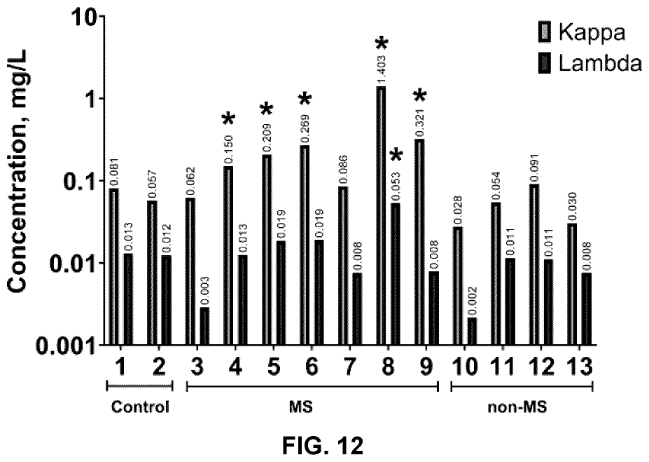

CSF was digested with LysC and analyzed with Exploris-240 as descried in Example 6. The absolute quantification of the kappa and lambda FLC dimers was done by spiking-in the known amounts of kappa and lambda SIL dimeric peptides SFNRGEC and TVAPTECS, respectively. FIG. 12 shows the concentrations of kappa and lambda dimers in CSF from healthy individuals, patients with multiple sclerosis and non-MS neurological conditions. The concentration of kappa FLC dimer in control subjects ranged from 0.057 to 0.081 mg/L. Five out of 7 (70%) of analyzed CSF samples from patients with multiple sclerosis had kappa FLC dimers concentration 1.85-fold to 17.3-fold higher than in control CSF. The amount of kappa dimers was slightly lower in subjects with non-MS neurological conditions. One of the two MS patients with low kappa FLC dimer concentration (patient #3) had the disease over a long period of time (over 25 years) and was treated with the disease-modifying drug (Ocrevus), which may have suppressed the production of kappa and lambda FLCs. The highest amount of kappa FLC dimers (1.4 mg/L) was detected in newly diagnosed patient #8. Interestingly, patient #8 also had lambda FLC dimer concentration of 0.053 mg/L, the highest among all subjects analyzed. Overall, lambda FLC dimer concentration in CSF of MS patients was comparable to that in control CSF (FIG. 12).

In conclusion, the FLC dimer assay is suitable for measuring the amount of kappa and lambda FLC dimers in the CSF of control and diseased subjects. The data in this example point out to the possibility of distinguishing MS patients from healthy controls and patients with other neurological conditions based on the amount of kappa FLC dimers in CSF.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Nakano T, Matsui M, Inoue I, Awata T, Katayama S, Murakoshi T. Free immunoglobulin light chain: its biology and implications in diseases. Clin Chim Acta 2011; 412 (11-12): 843-9 doi 10.1016/j.cca.2011.03.007.
2. Katzmann J A, Clark R J, Abraham R S, Bryant S, Lymp J F, Bradwell A R, et al. Serum Reference Intervals and Diagnostic Ranges for Free κ and Free λ Immunoglobulin Light Chains: Relative Sensitivity for Detection of Monoclonal Light Chains. Clinical Chemistry 2002; 48 (9): 1437-44.
3. Hampson J, Turner A, Stockley R. Polyclonal free light chains: promising new biomarkers in inflammatory disease. Current Biomarker Findings 2014 doi 10.2147/cbf.S57681.
4. Aggarwal R, Sequeira W, Kokebie R, Mikolaitis R A, Fogg L, Finnegan A, et al. Serum free light chains as biomarkers for systemic lupus erythematosus disease activity. Arthritis Care Res (Hoboken) 2011; 63 (6): 891-8 doi 10.1002/acr.20446.
5. Kraneveld A D, Kool M, van Houwelingen A H, Roholl P, Solomon A, Postma D S, et al. Elicitation of allergic asthma by immunoglobulin free light chains. Proc Natl Acad Sci 2005; 102 (5): 1578-83.
6. Bibasa M, Lorenzinia P, Cozzi-Leprib A, Calcagnoc A, di Giambenedettod S, Costantinie A, et al. Polyclonal serum-free light chains elevation in HIV-infected patients. AIDS 2012; 26 (16): 2107-10.
7 Dispenzieri A, Katzmann J A, Kyle R A, Larson D R, Therneau T M, Colby C L, et al. Use of nonclonal serum immunoglobulin free light chains to predict overall survival in the general population. Mayo Clin Proc 2012; 87 (6): 517-23 doi 10.1016/j.mayocp.2012.03.009.
8. Kumar S K, Rajkumar V, Kyle R A, van Duin M, Sonneveld P, Mateos M V, et al. Multiple myeloma. Nat Rev Dis Primers 2017; 3:17046 doi 10.1038/nrdp.2017.46.
9. Fotiou D, Theodorakakou F, Kastritis E. Biomarkers in A L Amyloidosis. Int J Mol Sci 2021; 22 (20) doi 10.3390/ijms222010916.
10. Dispenzieri A, Kyle R, Merlini G, Miguel J S, Ludwig H, Hajek R, et al. International Myeloma Working Group guidelines for serum-free light chain analysis in multiple myeloma and related disorders. Leukemia 2009; 23 (2): 215-24 doi 10.1038/leu.2008.307.
11. Kaplan B, Livneh A, Sela B A. Immunoglobulin free light chain dimers in human diseases. Scientific World Journal 2011; 11:726-35 doi 10.1100/tsw.2011.65.
12. Schiffer M. Molecular anatomy and the pathological expression of antibody light chains. Am J Pathol 1996; 148:1339-44.
13. Kaplan B, Golderman S, Aizenbud B, Esev K, Kukuy O, Leiba M, et al. Immunoglobulin-free light chain monomer-dimer patterns help to distinguish malignant from premalignant monoclonal gammopathies: a pilot study. Am J Hematol 2014; 89 (9): 882-8 doi 10.1002/ajh.23773.
14. Basile U, Gulli F, Gragnani L, Napodano C, Pocino K, Rapaccini G L, et al. Free light chains: Eclectic multipurpose biomarker. J Immunol Methods 2017; 451:11-9 doi 10.1016/j.jim.2017.09.005.
15. Gatt M E, Kaplan B, Yogev D, Slyusarevsky E, Pogrebijski G, Golderman S, et al. The use of serum free light chain dimerization patterns assist in the diagnosis of AL amyloidosis. Br J Haematol 2018; 182 (1): 86-92 doi 10.1111/bjh.15387.
16. Thompson A J, Banwell B L, Barkhof F, Carroll W M, Coetzee T, Comi G, et al. Diagnosis of multiple sclerosis: 2017 revisions of the McDonald criteria. The Lancet Neurology 2018; 17 (2): 162-73 doi 10.1016/s1474-4422 (17) 30470-2.
17. Kaplan B, Ganelin-Cohen E, Golderman S, Livneh A. Diagnostic utility of kappa free light chains in multiple sclerosis. Expert Rev Mol Diagn 2019; 19 (4): 277-9 doi 10.1080/14737159.2019.1586535.
18. Kaplan B, Golderman S, Yahalom G, Yeskaraev R, Ziv T, Aizenbud B M, et al. Free light chain monomer-dimer patterns in the diagnosis of multiple sclerosis. J Immunol Methods 2013; 390 (1-2): 74-80 doi 10.1016/j.jim.2013.01.010.
19. Kaplan B, Golderman S, Ganelin-Cohen E, Miniovitch A, Korf E, Ben-Zvi I, et al. Immunoglobulin free light chains in saliva: a potential marker for disease activity in multiple sclerosis. Clin Exp Immunol 2018; 192 (1): 7-17 doi 10.1111/cei.13086.
20. Lotan I, Ganelin-Cohen E, Tartakovsky E, Khasminsky V, Hellmann M A, Steiner I, et al. Saliva immunoglobulin free light chain analysis for monitoring disease activity and response to treatment in multiple sclerosis. Mult Scler Relat Disord 2020; 44:102339 doi 10.1016/j.msard.2020.102339.
21. Sarto C, Intra J, Fania C, Brivio R, Brambilla P, Leoni V. Monoclonal free light chain detection and quantification: Performances and limits of available laboratory assays. Clin Biochem 2021; 95:28-33 doi 10.1016/j.clinbiochem.2021.05.006.
22. Caponi L, Koni E, Romiti N, Paolicchi A, Franzini M. Different immunoreactivity of monomers and dimers makes automated free light chains assays not equivalent. Clin Chem Lab Med 2018; 57 (2): 221-9 doi 10.1515/cclm-2018-0412.
23. Agnello L, Lo Sasso B, Salemi G, Altavilla P, Pappalardo E M, Caldarella R, et al. Clinical Use of kappa Free Light Chains Index as a Screening Test for Multiple Sclerosis. Lab Med 2020; 51 (4): 402-7 doi 10.1093/labmed/Imz073.
24. Rajkumar S V, Dimopoulos M A, Palumbo A, Blade J, Merlini G, Mateos M-V, et al. International Myeloma Working Group updated criteria for the diagnosis of multiple myeloma. The Lancet Oncology 2014; 15 (12): e538-e48 doi 10.1016/s1470-2045 (14) 70442-5.
25. Messiaen A S, De Sloovere M M W, Claus P E, Vercammen M, Van Hoovels L, Heylen O, et al. Performance Evaluation of Serum Free Light Chain Analysis: Nephelometry vs Turbidimetry, Monoclonal vs Polyclonal Reagents. Am J Clin Pathol 2017; 147 (6): 611-22 doi 10.1093/ajcp/aqx037.
26. Moreau C, Lefevre C R, Decaux O. How to quantify monoclonal free light chains in plasma cell disorders: which mass spectrometry technology? Ann Transl Med 2020; 8 (15): 973 doi 10.21037/atm.2020.03.200.
27. Bergen H R, 3rd, Abraham R S, Johnson K L, Bradwell A R, Naylor S. Characterization of amyloidogenic immunoglobulin light chains directly from serum by on-line immunoaffinity isolation. Biomed Chromatogr 2004; 18 (3): 191-201 doi 10.1002/bmc.323.
28. Barnidge D R, Dispenzieri A, Merlini G, Katzmann J A, Murray D L. Monitoring free light chains in serum using mass spectrometry. Clin Chem Lab Med 2016; 54 (6): 1073-83 doi 10.1515/cclm-2015-0917.
29. Sepiashvili L, Kohlhagen M C, Snyder M R, Willrich MAV, Mills J R, Dispenzieri A, et al. Direct Detection of Monoclonal Free Light Chains in Serum by Use of Immunoenrichment-Coupled MALDI-TOF Mass Spectrometry. Clin Chem 2019; 65 (8): 1015-22 doi 10.1373/clinchem.2018.299461.
30. VanDuijn M M, Jacobs J F, Wevers R A, Engelke U F, Joosten I, Luider T M. Quantitative measurement of immunoglobulins and free light chains using mass spectrometry. Anal Chem 2015; 87 (16): 8268-74 doi 10.1021/acs.analchem.5b01263.
31. Mastroianni-Kirsztajn G, Nishida S K, Pereira A B. Are urinary levels of free light chains of immunoglobulins useful markers for differentiating between systemic lupus erythematosus and infection? Nephron Clin Pract 2008; 110 (4): c258-63 doi 10.1159/000167874.
32. Fan S B, Meng J M, Lu S, Zhang K, Yang H, Chi H, et al. Using pLink to Analyze Cross-Linked Peptides. Curr Protoc Bioinformatics 2015; 49:8 21 1-8 19 doi 10.1002/0471250953.bi0821s49.
33. MacLean B, Tomazela D M, Shulman N, Chambers M, Finney G L, Frewen B, et al. Skyline: an open source document editor for creating and analyzing targeted proteomics experiments. Bioinformatics 2010; 26 (7): 966-8 doi 10.1093/bioinformatics/btq054.
34. Kumar S, Dispenzieri A, Lacy M Q, Hayman S R, Buadi F K, Colby C, et al. Revised prognostic staging system for light chain amyloidosis incorporating cardiac biomarkers and serum free light chain measurements. J Clin Oncol 2012; 30 (9): 989-95 doi 10.1200/JCO.2011.38.5724.
35. Chahin M, Branham Z, Fox A, Leurinda C, Keruakous A R. Clinical Considerations for Immunoparesis in Multiple Myeloma. Cancers (Basel) 2022; 14 (9) doi 10.3390/cancers 14092278

1. A method for detection of immunoglobulin free light chain (FLC) dimers in a sample comprising:
(a) subjecting the sample to proteolytic digestion under non-reducing conditions, thereby obtaining a digested sample; and
(b) subjecting the digested sample to mass spectrometry analysis to detect immunoglobulin free light chain dimer peptides, wherein the detection of immunoglobulin free light chain dimer peptides is indicative of the presence of immunoglobulin free light chain dimers in the sample.

2. The method of claim 1, wherein the sample is a serum sample, cerebrospinal fluid sample or saliva sample.

3. (canceled)

4. The method of claim 1, wherein the immunoglobulin free light chain dimers comprise dimers of kappa light chains.

5. The method of claim 1, wherein the immunoglobulin free light chain dimers comprise dimers of lambda light chains.

6. The method of claim 1, wherein subjecting the sample to proteolytic digestion comprises contacting the sample with at least one endoprotease.

7. The method of claim 6, wherein the at least one endoprotease comprises trypsin, chymotrypsin, LysC, LysargiNase, or any combination thereof.

8. The method of claim 1, wherein the method further comprises subjecting the sample to a denaturating step prior to the proteolytic digestion and/or contacting the sample with a cysteine-modifying agent prior to the proteolytic digestion.

9. The method of claim 8, wherein the denaturating step comprises contacting the sample with urea.

10-11. (canceled)

12. The method of claim 8, wherein the cysteine-modifying agent comprises N-ethylmaleimide (NEM).

13. The method of claim 1, wherein the mass spectrometry is liquid chromatography coupled to parallel reaction monitoring.

14. (canceled)

15. The method of claim 1, wherein the immunoglobulin FLC dimers are measured by spiking in a known amount of synthetic peptides containing a specific label.

16. The method of claim 15, wherein the specific label comprises a heavy isotope-labeled amino acids.

17. The method of claim 1, further comprising enriching the sample in kappa and/or lambda light chains prior to subjecting the sample to proteolytic digestion.

18. The method of claim 17, wherein said enriching comprises contacting the sample with one or more matrices that bind the kappa and/or lambda light chains; and eluting the kappa and/or lambda light chains bound to the one or more matrices.

19. The method of claim 1, wherein the immunoglobulin free light chain dimer peptides are

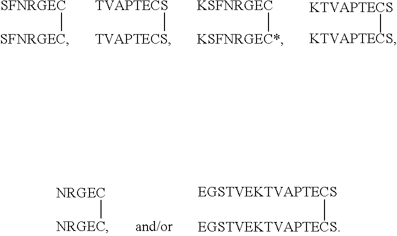

20. The method of claim 1, wherein the sample is a biological sample from a subject suffering from a disease or condition associated with the production of FLCs.

21. The method of claim 20, wherein the disease or condition is an autoimmune disease, an inflammatory condition, a viral infection, or a plasma cell dyscrasia (PCD).

22. (canceled)

23. The method of claim 20, wherein the sample is a serum sample from a human subject with multiple myeloma.

24. (canceled)

25. The method of claim 20, wherein the sample is (a) from a human subject suffering from or suspected of suffering from multiple sclerosis and/or a cerebrospinal fluid sample.

26. (canceled)

27. The method of claim 25, wherein the method comprises comparing the amount of immunoglobulin FLC dimers in the sample to the amount of immunoglobulin FLC dimers in a control sample from a healthy subject.
